(19) United States
(12) Patent Application Publication
(10) Pub. No.: US 2024/0207232 A1
(43) Pub. Date: Jun. 27, 2024

(54) METHOD OF TREATING A PATIENT DIAGNOSED WITH AN INTERSTITIAL LUNG DISEASE

(71) Applicants: Vicore Pharma AB, Stockholm (SE); Alex Therapeutics AB, Stockholm (SE)

(72) Inventors: Johan RAUD, Bromma (SE); Carl-Johan DALSGAARD, Nacka (SE); Jessica SHULL, Barcelona (ES); Kimmy BOLKE, Stockholm (SE); Oliver FLEETWOOD, Stockholm (SE)

(73) Assignees: Vicore Pharma AB, Stockholm (SE); Alex Therapeutics AB, Stockholm (SE)

(21) Appl. No.: 18/394,422

(22) Filed: Dec. 22, 2023

Related U.S. Application Data
(63) Continuation-in-part of application No. 18/352,807, filed on Jul. 14, 2023.
Application No. 18/352,807 is a continuation-in-part of application No. 18/173,882, filed on Feb. 24, 2023.

(30) Foreign Application Priority Data
Dec. 22, 2022 (GB) 2219591.1

(51) Int. Cl. A61K 31/4178; A61B 5/00; A61B 5/08; A61B 5/16; A61K 31/138; A61K 31/4418; A61K 31/4439; A61K 31/454; A61K 31/496; A61K 31/506; A61K 31/5513; A61K 31/5575; A61K 39/395; A61P 11/00; A61P 25/22; G16H 20/10; G16H 50/30

(52) U.S. Cl. CPC A61K 31/4178; A61B 5/08; A61B 5/165; A61B 5/4848; A61B 5/4884; A61K 31/138; A61K 31/4418; A61K 31/4439; A61K 31/454; A61K 31/496; A61K 31/506; A61K 31/5513; A61K 31/5575; A61K 39/3955; A61P 11/00; A61P 25/22; G16H 20/10; G16H 50/30

(57) ABSTRACT

Aspects of the present disclosure are directed to methods of treating a patient diagnosed with an interstitial lung disease (ILD), comprising administering a pharmaceutical drug for treating the ILD in a treatment regimen effective to treat fibrosis, inflammation, and/or vasculopathy, and in conjunction with the administration of the pharmaceutical drug, engaging in a self-directed digital therapeutic program that treats patient anxiety relating to the administration of the pharmaceutical drug, wherein the engaging in the self-directed digital therapeutic program is effective to increase adherence to the treatment regimen. Suitable pharmaceutical treatments include those that are useful in the therapy of an ILD, including pharmaceutical treatments that are in some way disease-modifying and/or capable of altering the course or the pathology of the ILD or at least slowing its progression, such as such as an antifibrotic agent, an immunomodulatory imide drug (IMID) or an angiotensin II type 2 receptor agonist (ATRAG).

### CROSS-REFERENCE TO RELATED APPLICATION

This application is a continuation-in-part of U.S. patent application Ser. No. 18/352,807, filed Jul. 14, 2023, which is a continuation-in-part of U.S. patent application Ser. No. 18/173,882, filed Feb. 24, 2023, which claims priority to United Kingdom Patent Application No. 2219591.1, filed Dec. 22, 2022, the entire content of both of which are incorporated herein by reference in their entireties.

### BACKGROUND

The term interstitial lung disease (ILD) refers to a large and heterogeneous group of parenchymal lung disorders characterized by damage to the lung tissue caused by various degrees of fibrosis and/or inflammation. The term used and recognised by patients with ILDs that have progressed to the fibrotic stage, is pulmonary fibrosis (PF). Each of the fibrosing ILDs is characterised by a chronic, often progressive, irreversible, and devastating scarring of the interstitial tissue in the lung and for the great majority of these diseases there is no existing cure.

There are more than 300 ILDs with similar symptoms of breathlessness, usually caused by reduced oxygen exchange in the alveoli. Approximately 65% of patients are affected by an ILD of unknown cause, which include idiopathic pulmonary fibrosis (IPF) and other idiopathic interstitial pneumonias (IIP), ILDs associated with connective tissue disorders such as rheumatoid arthritis (RA), systemic sclerosis (SSc); and sarcoidosis.

Known causes for ILDs include pneumoconiosis (asbestosis, silicosis), hypersensitivity pneumonitis and post-infectious ILD (ERS Whitebook). The distribution of ILDs shows large variation geographically, but studies in North America have generally shown ILD related to connective tissue disorder to be the most common ILD subtypes (Ley, B., Collard, H. R., & King, T. E., Jr. (2011), “Clinical course and prediction of survival in idiopathic pulmonary fibrosis”, Am J Respir Crit Care Med, 183(4), 431-440. https://doi.org/10.1164/rccm.201006-0894CI). IPF is practically always associated with progressive pulmonary fibrosis, but patients diagnosed with other ILDs are also at risk of developing progressive pulmonary fibrosis, often with a phenotype not dissimilar to IPF. Although the underlying disease may in some cases be managed by medication, the development of progressive pulmonary fibrosis implies a non-remitting course of disease, associated with an increasing risk of morbidity and mortality.

IPF is characterised by a progressive deposition of extracellular matrix proteins and fibrous tissue in the lungs, resulting in destruction of lung architecture and reduced lung capacity. Symptoms of IPF, as with other types of PF, include shortness of breath, fatigue, and a dry intractable cough. IPF is lethal, and death is most commonly caused by acute or subacute respiratory failure due to progression of lung fibrosis. The prognosis of IPF is poor, with an estimated life expectancy of 3-5 years after diagnosis which is shorter than many malignancies. The prognosis for the other diseases leading to PF varies.

There are presently no curative treatment options except, in rare cases, lung transplantation. IPF results in a chronic, irreversible, progressive deterioration in lung function and then, in most cases, death within 3-5 years. While the overall prognosis is poor in IPF, it is difficult to predict the rate of progression in individual patients. Risk factors for IPF include age, male gender, genetic predisposition and history of cigarette smoking. The annual incidence is between 5-16 per 100,000 individuals, with a prevalence of 13-20 cases per 100,000 people, increasing dramatically with age (King et al., Lancet (2011) 378, 1949-1961; Noble et al., J. Clin. Invest. (2012) 122, 2756-2762). IPF s recalcitrant to therapies that target the immune system which distinguishes it from pulmonary fibrosis (PF) associated with other, purely systemic diseases.

The age- and sex-adjusted prevalence of fibrosing ILD per 100,000 in the US population has been estimated at approximately 118, of which about 70 were assessed to have chronic fibrosing ILD with a progressive phenotype (Olson A L, Patnaik P, Hartmann N, Bohn R L, Garry E M, Wallace L, “Prevalence and Incidence of Chronic Fibrosing Interstitial Lung Diseases with a Progressive Phenotype in the United States Estimated in a Large Claims Database Analysis”, Adv Ther, 2021 July; 38(7): 4100-4114, doi: 10.1007/s12325-021-01786-8. Epub 2021 Jun. 17. PMID: 34156606; PMCID: PMC8279991).

Specifically looking at IPF, there are approximately 3 million people worldwide living with the disease. Debilitating symptoms typically appear between the ages of 50 and 70 years and, while the disease is most common in men, the number of cases in women is increasing. With PF related to connective tissue disorder or sarcoidosis, onset usually starts much earlier in life (30-50) and affects women as well as men.

At present there are no medicines that cure or even improve the pulmonary fibrosis associated with ILDs. There are two antifibrotic drugs, nintedanib and pirfenidone, which are approved for treatment of IPF. Nintedanib is additionally approved for treatment of chronic fibrosing ILDs with a progressive phenotype and for slowing the rate of decline in pulmonary function in patients with systemic sclerosis-associated ILD (SSC-ILD). Both nintedanib and pirfenidone merely slow disease progression (annual rate of decline of FVC) and are not a long-term cure, while having significant, primarily gastrointestinal, side effects.

The knowledge of being diagnosed with an ILD, such as IPF, comes as a shock to many patients as there is often no identified cause and patients may feel that they lose control of living the way they want. They fear that they will no longer be able to fulfil their role in life, whether that be as a parent, spouse, or grandparent. In the case of IPF, this is often at a time in life when patients are approaching retirement. People with an ILD learn that daily activities may have to change; normal routines like taking walks or travel must be adjusted around the debilitating respiratory symptoms of the disease, as well as the use of oxygen tanks and tubes, safety measures for protection against infections like COVID-19, and commonly a persistent and embarrassing cough. This can have a negative impact on mental health, particularly in patients experiencing a noticeable deterioration in physical symptoms. Several studies have reported that symptoms of depression and anxiety are common in patients with IPF. The prevalence of depression ranges from 24.3-49.2% while the proportion of patients with anxiety may be as high as 60% in IPF (Akhtar, A. A., Ali, M. A., & Smith, R. P. (2013), “Depression in patients with idiopathic pulmonary fibrosis”, Chron Respir Dis, 10(3), 127-133, https://doi.org/10.1177/1479972313493098; Glaspole, I. N., Watson, A. L., Allan, H., Chapman, S., Cooper, W. A., Corte, T. J., Ellis, S., Grainge, C., Goh, N., Hopkins, P., Keir, G., Macansh, S., Mahar, A., Moodley, Y., Reynolds, P. N., Ryerson, C. J., Walters, E. H., Zappala, C. J., & Holland, A. E. (2017), “Determinants and outcomes of prolonged anxiety and depression in idiopathic pulmonary fibrosis”, Eur Respir J, 50(2), https://doi.org/10.1183/13993003.00168-2017; Ryerson, C. J., Berkeley, J., Carrieri-Kohlman, V. L., Pantilat, S. Z., Landefeld, C. S., & Collard, H. R. (2011), “Depression and functional status are strongly associated with dyspnea in interstitial lung disease”, Chest, 139(3), 609-616, https://doi.org/10.1378/chest.10-0608). Studies have also shown that IPF reduces quality of life (Lee, Y. J., Choi, S. M., Lee, Y. J., Cho, Y. J., Yoon, H. I., Lee, J. H., Lee, C. T., & Park, J. S. (2017), “Clinical impact of depression and anxiety in patients with idiopathic pulmonary fibrosis”, PloS One, 12(9), e0184300, https://doi.org/10.1371/journal.pone.0184300). Several studies indicate that the problem with anxiety is applicable to ILDs in general, and not limited to IPF (Abebaw Mengistu Yohannes, “Depression and anxiety in patients with interstitial lung disease”, Expert Review of Respiratory Medicine, 14(9) 859-862, https://doi.org/10.1080/17476348.2020.1776118).

Cognitive behavioural therapy (CBT) is a recommended first-line treatment for anxiety disorders (Powers, M. B., E.; Gorman, J.; Kissen, D.; Smits, J; (2015), “Anxiety and Depression Association of America—Clinical practice review for GAD”, https://adaa.org/resources-professionals/practice-guidelines-gad). However, the usual standard of care for IPF in the US does not include referral to a psychologist, despite the potential benefits of treatment (Raghu, G., Rochwerg, B., Zhang, Y., Garcia, C. A., Azuma, A., Behr, J., Brozek, J. L., Collard, H. R., Cunningham, W., Homma, S., Johkoh, T., Martinez, F. J., Myers, J., Protzko, S. L., Richeldi, L., Rind, D., Selman, M., Theodore, A., Wells, (2015) “An Official ATS/ERS/JRS/ALAT Clinical Practice Guideline: Treatment of Idiopathic Pulmonary Fibrosis. An Update of the 2011 Clinical Practice Guideline”, Am J Respir Crit Care Med, 192(2), e3-19, https://doi.org/10.1164/rccm.201506-1063ST).

Patients with an ILD face barriers in accessing traditional CBT as it can be very expensive, and the costs are typically not reimbursed or only minimally reimbursed. However, even if cost were not an issue, there are not enough specialists to serve the need. In addition, there are difficulties/risks associated with travelling to a psychologist's office, whether due to risk of infections like COVID-19 or distance. Studies have shown that patients with chronic lung diseases including IPF have poor clinical outcomes with COVID-19 disease (Gerayeli, F. V., Milne, S., Cheung, C., Li, X., Yang, C. W. T., Tam, A., Choi, L. H., Bae, A., & Sin, D. D. (2021), COPD and the risk of poor outcomes in COVID-19: A systematic review and meta-analysis, EclinicalMedicine, 33, 100789, https://doi.org/10.1016/j.eclinm.2021.100789; Naqvi, S. F., Lakhani, D. A., Sohail, A. H., Maurer, J., Sofka, S., Sarwari, A., & Hadi, Y. B. (2021), Patients with idiopathic pulmonary fibrosis have poor clinical outcomes with COVID-19 disease: a propensity matched multicentre research network analysis, BMJ Open Respir Res, 8(1). https://doi.org/10.1136/bmjresp-2021-000969). In fact, any airway infection can cause exacerbations of chronic lung diseases. As a result, it is therefore likely that patients with IPF and other ILDs are less willing to have face-to-face counselling.

### SUMMARY OF THE INVENTION

Aspects of the present disclosure are directed to methods of treating an interstitial lung disease (ILD) in a patient, comprising administering a pharmaceutical treatment that is useful in the therapy of an ILD; and, in conjunction with the administration of the pharmaceutical treatment, engaging in a digital therapeutic program that delivers cognitive behavioural therapy for the treatment of psychological, behavioural, and/or physical aspects of living with the ILD.

A method of treating a patient diagnosed with an interstitial lung disease (ILD) may comprise administering a pharmaceutical drug for treating the ILD in a treatment regimen effective to treat fibrosis, inflammation, and/or vasculopathy, and in conjunction with the administration of the pharmaceutical drug, engaging in a self-directed digital therapeutic program that treats patient anxiety relating to the administration of the pharmaceutical drug, wherein the engaging in the self-directed digital therapeutic program is effective to increase adherence to the treatment regimen.

Suitable pharmaceutical treatments include those that are useful in the therapy of an ILD, including pharmaceutical treatments that are in some way (IL) disease-modifying and/or capable of altering the course or the pathology of the ILD or at least slowing its progression, such as such as an antifibrotic agent, an immunomodulatory imide drug (IMID) or an angiotensin II type 2 receptor agonist (ATRAG), such as C21 (defined below). Suitable pharmaceutical treatments also include concomitant treatments for diseases that are associated with ILDs, including those that treat a comorbidity of an ILD, or are capable of treating sequelae of ILDs, including mental and/or psychosomatic effects or exacerbations.

In some embodiments, the pharmaceutical treatment may be selected from anti-inflammatory, an anti-tussive, an immunomodulatory, a cytotoxic, an anti-oxidant, an anti-coagulant, an anti-chemokine, or an anti-angiogenic drug.

In some embodiments, the pharmaceutical treatment may be a RAS-blocker, an endothelin antagonist or sildenafil.

In some embodiments, the pharmaceutical treatment may be selected from one or more antifibrotic agents, such as nintedanib, pirfenidone, immunomodulatory imide drugs (IMIDs) or an angiotensin II type 2 receptor agonist (ATRAG).

In some embodiments, the pharmaceutical treatments include anti-tussive drugs (e.g. dextromethorphan, codeine, benzonate and nalbuphine), glucocorticoids (e.g., beclomethasone, betamethasone, budesonide, cortisone, dexamethasone, hydrocortisone, methylprednisolone, prednisolone, prednisone and triamcinolone); the c-Jun N-terminal kinase inhibitor CC-90001 (Celgene/Bristol Myers Squibb); the HSP47 RNAi therapeutic ND-L02-s0201/BMS-986263 (Nitto Denko/Bristol Meyers Squibb), the Hedgehog cell-signalling pathway inhibitor ENV-101/taladegib (Endeavor Biomedicines), the dual inhibitor of αVβ1/αVβ6 PLN-74809 (Pliant Therapeutics), the dual inhibitor of NADPH oxidase isoforms NOX4 and NOX1 setanaxib/GKT137831/GKT831 (Calliditas Therapeutics/Genkyotex), HEC585 (Sunshine Lake Pharma Co), the Galectin-3 inhibitor GB0139/TD139 (Galecto Biotech), Src tyrosine kinase inhibitor saracatinib (AstraZeneca), the humanised anti-FXIIa monoclonal antibody garadacimab/CSL312 (CSL Behring). The LPA1 receptor antagonist BMS-986278 (Bristol Myers Squibb), the LPA1 receptor antagonist HZN-825 (Horizon Therapeutics), the deuterium-substituted analogue of pirfenidone deupirfenidone/LYT100 (PureTech), the autotaxin inhibitor cudetaxestat/BLD-0409 (Blade Therapeutics), the prolyl-tRNA synthetase inhibitor DWN12088 (Daewoong Pharmaceutical Co), the monoclonal antibody AMB-05X targeting colony stimulating factor 1 receptor (AmMax Bio), the IL-11 receptor-blocking antibody LASN01 (Lassen Therapeutics), the MMP7-lowering RNAi treatment ARO-MMP7 (Arrowhead Pharmaceuticals), the autotaxin inhibitor BBT-877 (Bridge Biotherapeutics), the smad ubiquitin regulatory factor 1 inhibitor LTP001 (Novartis), AP-01/inhaled pirfenidone (Avalyn Pharma), tipelukast/MN-001 (MediciNova/Kyorin), Ifenprodil/NP-120 (Algernon Pharmaceuticals), RG-6354/PRM-151/recombinant human pentraxin-2 (Roche/Promedior), the prostacyclin analogue treprostinil (United Therapeutics), the phosphodiesterase 4 inhibitor BI 1015550 (Boehringer Ingelheim; [1-[[(5R)-2-[4-(5-chloropyrimidin-2-yl)piperidin-1-yl]-5-oxo-6,7-dihydrothieno[3,2-d]pyrimidin-4-yl]-amino]cyclobutyl]methanol; see, for example, Maher et al, ERJ Open Research (2022) 8: 00240-2022; doi: 10.1183/23120541.00240-2022); xylocaine; inhaled nitric oxide; other immunosuppressants, such as azathioprine and mycophenolate mofetil; vitamins (e.g. vitamin B, C and D); mucolytics (e.g. acetylcysteine and ambroxol); inflammation suppressants, such as cyclophosphamide; other immunosuppressants, such as azathioprine and mycophenolate mofetil; and antioxidants, such as N-acetylcysteine.

Preferred pharmaceutical treatments include, antifibrotics (e.g., nintedanib and pirfenidone); IMIDs (e.g. lenalidomide, pomalidomide and, particularly, thalidomide) and, most preferably, ATRAGs, such as any of those described generically and specifically in international patent applications with publications WO 2021/053344, WO 2021/186185, WO 2021/186180, WO 2021/186180, WO 2022/200787, WO 2022/200786 and WO 2022/200785, unpublished international patent application PCT/GB2022/051760 and, especially, N-butyloxycarbonyl-3-(4-imidazol-1-ylmethylphenyl)-5-iso-butylthiophene-2-sulfonamide (Compound 21 or ‘C21’).

In a preferred embodiment, the pharmaceutical treatment is N-butyloxycarbonyl-3-(4-imidazol-1-ylmethylphenyl)-5-iso-butylthiophene-2-sulfonamide (Compound 21 or ‘C21’).

In some embodiments, the pharmaceutical treatment may be an anxiolytic drug. Suitable anxiolytic drugs include selective serotonin reuptake inhibitors including, but not limited to, citalopram, escitalopram, fluoxetine, fluvoxamine, paroxetine, sertraline and dapoxetine; serotonin-norepinephrine reuptake inhibitors including, but not limited to, desvenlafaxine, duloxetine, levomilnacipran, milnacipran, sibutramine, tramado and venlafaxine; benzodiazepines including, but not limited to, clorazepate, diazepam, flurazepam, halazepam, prazepam, lorazepam, lormetazepam, oxazepam, temazepam, clonazepam, flunitrazepam, nimetazepam, nitrazepam, adinazolam, alprazolam, estazolam, triazolam, climazolam, loprazolam, midazolam and clobazam; antipsychotic drugs including, but not limited to, trifluoperazine, quetiapine, aripiprazole, olanzapine and risperidone; beta-adrenoceptor antagonists including, but not limited to, acebutolol, bisoprolol, carvedilol, propranolol, atenolol and metoprolol; and other anxiolytic drugs such as buspirone, carbamazepine, pregabalin, gabapentin, lamictal, tegretol.

Such pharmaceutical treatments may be administered alone or in combination with each other and/or may be administered prior to and during the course of the digital therapeutic.

Pharmaceutically-acceptable doses, of pharmaceutical treatments such as those mentioned above include those that are known in the art and described for the drugs in question to in the medical literature, such as Martindale—The Complete Drug Reference, 38^{th} Edition, Pharmaceutical Press, London (2014) and the documents referred to therein, the relevant disclosures of which documents are hereby incorporated by reference.

When the pharmaceutical treatment comprises C21, suitable lower daily doses in adult patients (average weight e.g., 70 kg), may be about 10 mg, such as about 20 mg, for example about 25 mg, per day. Suitable upper limits of daily dose ranges of C21 may be about up to about 900 mg, such as 600 mg, including about 400 mg. In particular, daily doses may be about 300 mg, such as about 250 mg, including about 200 mg, such as about 175 mg or about 150 mg, including about 100 mg, and including about 75 mg, about 60 mg or about 50 mg.

All of the above doses are calculated as free C21. Doses may be split into multiple individual doses per day. Doses may be given between once and six, such as four times daily, preferably three times daily and more preferably twice daily.

In some embodiments, the method of treating an ILD in a patient comprises administering a pharmaceutical treatment for use in an ILD and, in conjunction with the administration of the pharmaceutical treatment, engaging in a digital therapeutic program that delivers cognitive behavioural therapy for the treatment of a psychological symptom of the ILD.

In some embodiments, the method of treating the ILD in a patient comprises: administering a pharmaceutical treatment for an ILD; and, in conjunction with the administration of the pharmaceutical treatment, engaging in a digital therapeutic program that delivers cognitive behavioural therapy, thereby achieving improved suppression and/or alleviation of symptoms of the relevant ILD compared with a patient receiving only pharmaceutical treatment (such as pirfenidone, nintedanib, buspirone, clonazepam, desvenlafaxine, fluoxetine, gabapentin, lorazepamomeprazole, pantoprazole and/or rabeprazole).

In some embodiments, the suppression or alleviation of the symptoms of the relevant ILD is statistically significant compared with a patient receiving only pharmaceutical treatment.

In some embodiments, the method of treating the relevant ILD in a patient comprises: administering a pharmaceutical treatment for ILD; and, in conjunction with the administration of the pharmaceutical treatment, engaging in a digital therapeutic program that delivers cognitive behavioural therapy for the treatment of the ILD thereby providing a more effective treatment.

Aspects of the present disclosure relate to a digital therapeutic for the treatment of symptoms of anxiety in a patient diagnosed with an interstitial lung disease (ILD) such as idiopathic pulmonary fibrosis (IPF) or any ILD causing pulmonary fibrosis (PF).

There is presently no product on the market specifically addressing the psychological aspects of living with an ILD, such as IPF. The digital therapeutic described herein was developed specifically to address this unmet need—providing an accessible, digital CBT therapy to specifically deal with the doubts, fears, and anxieties that ILD patients face.

Accordingly, in some embodiments there is provided a digital therapeutic intended for the treatment of symptoms of anxiety and improvement of health-related quality of life in patients with ILDs by providing neurobehavioral interventions from CBT and related disciplines. The digital therapeutic disclosed and described herein may be used adjunctively to other treatment modalities for ILDs, including IPF. The digital therapeutic is intended to treat and improve the symptoms and anxiety caused by an ILD but is not intended to directly treat an ILD or the physical manifestations of ILDs.

In some embodiments, there is provided a digital therapeutic for the treatment of a patient diagnosed with ILD, the digital therapeutic comprising computer executable instructions for: conducting a personalised functional analysis with the patient to determine a first personal pulmonary fibrosis (PF) signature, the first personal PF signature including one or more anxiety triggers which may be defined as one or more events that a particular patient identifies on a personal basis as exacerbating the symptoms of anxiety and thereby causing behavioural restrictions; storing the first personal PF signature; identifying one or more digital cognitive behavioural therapy (dCBT) tools for modifying the first personal PF signature; determining a first personalised dCBT treatment plan which incorporates the use of the identified dCBT tools; administering the first personalised dCBT treatment plan; and, determining changes in relation to the behavioural restrictions associated with the first personal PF signature of the patient.

In some embodiments, the digital therapeutic may further comprise computer executable instructions for determining the level of anxiety (and therefore behavioural restrictions) that may result from the one or more anxiety triggers before administering the first personalised dCBT treatment plan.

In some embodiments, the digital therapeutic may further comprise computer executable instructions for determining the level of anxiety (and therefore behavioural restrictions) that may result from the one or more anxiety triggers after administering the first dCBT treatment plan.

In some embodiments, the digital therapeutic may further comprise computer executable instructions for comparing the determined anxiety levels to assess changes in the personal PF signature of the patient.

In some embodiments, the dCBT tools for modifying the personal PF signature may be contained within one or more modules. The modules may be classified as information modules or therapy modules, and patient access to the therapy modules may be restricted.

In some embodiments, administering the first personal dCBT treatment plan comprises steps of: providing the patient with an exercise introduction incorporating one or more questions for the patient to answer, wherein the exercise introduction is related to an exercise that is part of an identified dCBT tool for modifying the first personal PF signature; in response to the answers provided by the patient, modifying the exercise; and providing the patient with the modified exercise.

In some embodiments, at least one of the identified dCBT tools comprises an exercise, and wherein after completion of the exercise the associated dCBT tool is unlocked for future use by the patient.

In some embodiments, determining changes in relation to the behavioural restrictions associated with the first personal PF signature of the patient further comprises a step of determining changes in the perceived quality of life of the patient.

In some embodiments, the digital therapeutic further comprises computer executable instructions for: determining a second personal PF signature, by modifying the first personal PF signature to incorporate the determined changes in relation to the behavioural restrictions associated with the first personal PF signature of the patient; and storing the second personal PF signature.

In some embodiments, the digital therapeutic further comprises computer executable instructions for: identifying one or more digital cognitive behavioural therapy (dCBT) tools for modifying the second personal PF signature; determining a second personalised dCBT treatment plan which incorporates the use of the identified dCBT tools; administering the second personalised dCBT treatment plan; and determining changes in relation to the behavioural restrictions associated with the second personal PF signature of the patient.

The digital therapeutic disclosed and described herein may be used to treat anxiety caused by any disease or condition with dyspnea/difficulty breathing as a symptom, including asthma, chronic obstructive pulmonary disease, emphysema, pulmonary hypertension such as pulmonary arterial hypertension, respiratory infections such as bronchitis and pneumonia, airway-blocking cancers including lung cancer, cardiac diseases such as heart failure and myocardial ischemia, neuromuscular disorders affecting the respiratory muscles, pregnancy, obesity and/or psychogenic breathing disorders.

### BRIEF DESCRIPTION OF THE DRAWINGS

An example of the present disclosure will now be described in detail with reference to the accompanying drawings, in which:

FIG. 1a illustrates a schematic diagram of a computer system for providing digital therapeutic treatment;

FIG. 1b illustrates a schematic block diagram of a user device for implementing a digital therapeutic;

FIG. 1c illustrates a schematic block diagram of functional units of a digital therapeutic;

FIG. 2 illustrates example steps for providing a digital therapeutic for the treatment of a patient diagnosed with pulmonary fibrosis;

FIG. 3 illustrates an example of a patient journey;

FIGS. 4-8 illustrate examples of a possible implementation of the digital therapeutic in the form of an app for use on a smartphone;

FIG. 9 illustrates example steps for providing acceptance and commitment therapy, including the use of exposure and an exposure ladder;

FIG. 10 illustrates and example of an exposure ladder, presented in the form of an app on a digital therapeutic; and,

FIG. 11 illustrates the results of a study of patients using a digital therapeutic app designed according to the present disclosure, showing significant improvement over control in each domain at week 9 and at 12-week follow-up.

### DETAILED DESCRIPTION

Patients with IPF usually seek medical assistance due to chronic and progressive exertional dyspnea and cough. Imaging of the lung classically reveals traction bronchiectasis, thickened interlobar septae, with (typical Usual Interstitial Pneumonia (UIP)) or without (possible UIP) subpleural honeycombing. When all three manifestations are present and there is no evidence of a systemic connective tissue disease or environmental exposure, a diagnosis of IPF is very likely. A definite diagnosis is usually made by high-resolution computed tomography (HRCT) and/or lung biopsy and requires a multidisciplinary team of expertise including pulmonologists, radiologists and pathologists experienced in ILDs.

IPF demonstrates different stages with different prognosis, defined as mild, moderate and severe. Mild cases follow a stable progressive path with patients sometimes taking several years to seek medical advice. Acute exacerbations of IPF are defined as a rapid worsening of the disease, and patients often deteriorate from mild to moderate or moderate to severe with very poor outcomes with a high mortality rate in the short run.

The cause of IPF is unknown but it appears to be a disorder likely arising from an interplay of environmental and genetic factors resulting in fibroblast driven unrelenting tissue remodelling rather than normal repair; a pathogenesis primarily driven by fibrosis rather than inflammation. A growing body of evidence suggests that the disease is initiated through alveolar epithelial cell microinjuries and apoptosis, activating a scarring process The fibroblastic foci secrete exaggerated amounts of extracellular matrix that destroys the lung parenchyma and ultimately leads to loss of lung function.

As a restrictive lung disease, IPF is typically diagnosed using static spirometry. Here, the only consideration is the volume of air that is exhaled, as opposed to dynamic spirometry, which measures the time taken to exhale a certain volume of air, and is typically used to diagnose obstructive lung diseases, like COPD and asthma.

The most usual and useful static spirometric test to diagnose IPF and/or monitor its progression is the forced vital capacity (FVC) test, in which a subject is urged to breathe in as far as he or she can, and then out as far as he or she can. This test is classified as static because it does not involve an element of time.

The mean annual rate of decline in lung function (FVC) in IPF is within a range of 0.13-0.4 litres. Symptoms precede diagnosis by 1-2 years and radiographic signs may precede symptoms (Ley et al., Am. J. Respir. Crit. Care Med. (2011) 183, 431-440).

Numerous IPF treatment approaches have been tested in pre-clinical models and clinical trials such as anti-inflammatory, immune-modulatory, cytotoxic, general anti-fibrotic, anti-oxidant, anti-coagulant, anti-chemokine, anti-angiogenic drugs as well as RAS-blockers, endothelin antagonists, and sildenafil, all of which have basically been shown to provide limited or no benefits (Rafii R et al., J. Thorac. Dis. (2013) 5, 48-73).

Current clinical treatment of IPF includes oxygen supplementation. Medications that are used include pirfenidone or nintedanib, but with only limited success in slowing the progression of the disease.

For example, the review article by Maher and Strek (Respiratory Research, 20, 205 (2019)) states that, clinical trials have demonstrated that nintedanib and pirfenidone reduce the decline in lung function in patients with IPF, more specifically reducing the rate of decline (according to FVC measurements) by approximately 50% over the period of a year.

Thus, although nintedanib and pirfenidone are believed to reduce the risk of acute deteriorations in lung function and to improve life expectancy by reducing the rate at which IPF progresses, and also (as reported by Isshiki et al, Respiratory Medicine, 187, 106551 (2021)) that they may help protect against acute exacerbation of IPF (AE-IPF; acute respiratory worsening in the absence of other known causes, which has a very poor median survival rate), both of these drugs are only capable of slowing down the progression of the disease. Furthermore, these drugs commonly cause (predominantly gastrointestinal) side-effects, which can sometimes curtail treatment (see e.g., Maher and Strek supra).

Currently, a lung transplant is the only intervention that substantially improves survival in IPF patients. However, complications such as infections and transplant rejection are not uncommon.

The Renin-Angiotensin System (RAS) is a key regulator of blood pressure homeostasis. Renin, a protease, cleaves its only known substrate (angiotensinogen) to form angiotensin I (Ang I), which in turn serves as substrate to angiotensin converting enzyme (ACE) to form Ang II. The endogenous hormone Ang II is a linear octapeptide (Asp¹-Arg²-Val³-Tyr⁴-Ile⁵-His⁶-Pro⁷-Phe⁸) and is an active component of the renin angiotensin system (RAS).

The angiotensin II type 1 (AT1) receptor is expressed in most organs and is believed to be responsible for the majority of the pathological effects of Ang II. The safety and efficacy of losartan (an AT1-receptor inhibitor) has recently been investigated in a small uncontrolled open-label pilot trial on IPF (www.clinicaltrials.gov identifier NCT00879879).

Several studies in adult individuals appear to demonstrate that, in the modulation of the response following Ang II stimulation, activation of the angiotensin II type 1 (AT2) receptor has opposing effects to those mediated by the AT1 receptor.

AT2 receptor agonists (ATRAGs) have been shown to be of potential utility in the treatment and/or prophylaxis of disorders of the alimentary tract, such as dyspepsia and irritable bowel syndrome, as well as multiple organ failure (see international patent application WO 99/43339).

N-Butyloxycarbonyl-3-(4-imidazol-1-ylmethylphenyl)-5-iso-butylthiophene-2-sulfonamide (Compound 21 or, as used hereinafter, ‘C21’) is a selective ATRAG. Promising findings in preclinical models of IPF (see e.g., Bruce et al, Br. J. Pharmacol., 172, 2219 (2015) and Rathinasabapathy et al, Front. Physiol., 9, 180 (2018)), led to C21 being in clinical development for use in IPF treatment (see also international patent application WO 2016/139475).

In an ongoing Phase II clinical trial in IPF patients, although the targeted efficacy endpoint at the outset of this study was a reduction in the deterioration in lung function, as measured by FVC, over time compared to current standard of care in IPF (nintedanib and pirfenidone), it was found, remarkably, that C21 is not only capable of arresting the progression of deterioration in lung function in IPF patients in a clinical setting, but actually at least partially restoring it, potentially representing an extremely important development in the treatment of this debilitating and ultimately fatal disease.

In addition to, and, to some extent, as a consequence of, the physical symptoms of IPF, anxiety and its associated mental health problems, such as depression, is a very common and serious comorbidity in people with idiopathic pulmonary fibrosis (IPF), which drastically affect quality of life.

To make matters worse, the anxiety associated with the anticipation of exhibiting IPF symptoms, including shortness of breath or a coughing fit, can often bring about the precise physiological response that results in such symptoms, which in turn can make the anxiety worse.

In this respect, there is a real clinical need, not only for safer and/or more effective treatments of ILDs, such as IPF, that deal not only with the physical symptoms mentioned above, but also more holistic and personalized treatments that deal with a patient's mental well-being. The development of new treatment strategies is very important, and a fundamental challenge for the future is to develop appropriate therapeutic approaches that will not only slow down or stop the progression of the disease, but also improve quality of life for those suffering from the condition.

Studies have also shown that anxiety symptoms significantly impact quality of life in patients with IPF. Several studies have also indicated that the problem with anxiety is applicable to ILDs in general, and is not limited to IPF. Anxiety and depression are associated with adverse consequences by impairing quality of life, increasing hospital admissions and length of hospital stays, increased exacerbations, poor adherence to medical treatment, dependency on caregivers, reduced social interactions, and contributing to premature death (Yohannes A M. Expert Rev Respir Med. (2020) September, 14(9):859-862; Glaspole et al. Eur Respir J. (2017) Aug. 17, 50(2): 1700168).

Left untreated, the anxiety triggers identified in the personal PF signature themselves can be exacerbated and/or propagated by a patient's emotional state at the relevant time, and in particular feelings of anxiety, fear, worry, uncertainty, and a feeling of helplessness and, accordingly, can lead to avoidance behaviour, thereby decreasing activity levels. Avoidance of activities, especially exercise and increasing blood flow, can be irreversibly debilitating in a population where oxygen uptake is notably deteriorating. Physical activity can be critical for maintaining vital functions, as can be socialisation and feeling needed. Many people with PF are aged 60 or older and may avoid seeing family and friends because of fears such as that their oxygen tank may run out, that they could catch a virus, or that they think family and friends may be uncomfortable hearing them cough.

As discussed in more detail below, the treatment method according to the present disclosure is structured so that the person undertaking the treatment will modify their behaviours. The mechanism of action is to influence the person to gradually ease into activities that are associated with one or more anxiety triggers identified in the personal PF signature. This may involve gradually undertaking one or more patient-identified activities, providing information and support as to how to continue doing these activities safely, documenting how it was and when they will repeat the activity. By gradually introducing the person into activities in this manner, the user is slowly “vaccinated” against the anxiety triggers, which may involve, for example, debilitating thoughts that stop activity. Increasing involvement in activities that involve one or more anxiety triggers, such as social and/or cardiovascular activities improves the Quality of Life (QoL) of the person.

QoL may be measured for the PF population using any suitable measurement method, such as the King's Brief Interstitial Lung Disease questionnaire (KBILD). KBILD is a validated health-related QoL measure used in clinical trials for PF patients globally. As discussed in more detail below, a digital therapeutic app designed according to the present disclosure has been tested in a further study involving 108 patients in which the QoL of the patients using the app was assessed using the KBILD questionnaire.

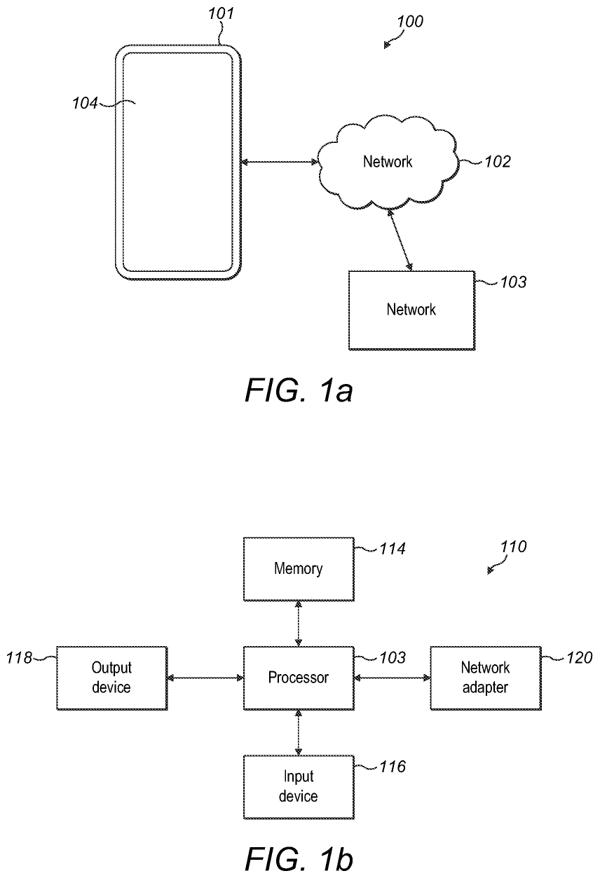

FIG. 1a illustrates a schematic diagram of a computer system for providing a digital therapeutic treatment. The system 100 comprises a user device 101 connected to a server 103 via a network 102. The user device 101 is configured to provide a digital therapy to a patient. The digital therapeutic system 100 may be utilised to implement one, some, or all of the techniques or features described herein. In one embodiment, the digital therapeutic system 100 can provide a patient access to the digital therapeutic 104 via a therapy application and facilitate or encourage interaction between the patient with the digital therapeutic 104. The digital therapeutic 104, or particular restricted features of a digital therapeutic 104, could be prescribed to the patient 101, for example by a healthcare professional (HCP).

The user device 101 may be implemented using any conventional computer system such as, for example, a laptop, cellular telephone, desktop computer or tablet computer. Having readily available access to digital CBT provided via the digital therapeutic system 100 enables improved patient outcomes by helping patients to manage the symptoms and anxiety relating to an ILD, such as IPF, and may improve their overall health and wellbeing, among other benefits.

The digital therapeutic may be accessed via a therapy application using the user device 101. For example, the digital therapeutic 104 may be rendered using a browser (for example in a web-based implementation of the therapy application) or a bespoke software application (for example a smartphone app). In particular, the program may be incorporated into an app accessible from a mobile phone or wearable device, which may also be used to drive further content for, and patient interaction with, the program. Alternatively, the therapy application may be stored and run remotely from the user device 101, within the network 102 or at the server 103. Similarly, data accessed by the therapy application may be stored locally at the user device 101, within the network 102 or at the server 103.

In some embodiments, the therapy application and its data to be stored locally at the user device 101. Data concerning the patient may be encrypted while stored locally and decrypted by the therapy application during use. The benefits of local storage specific to this application include: improve speed of data access, improve security by removing the need for network communication during use and so avoiding the transmission of sensitive data on potentially insecure networks, avoiding a single point of storage of sensitive data within the network that may be a target for malicious access, avoiding user concerns for privacy due to the knowledge that their interactions with the application are not provided to a healthcare provider or other entity. In examples in which the therapy application and data are stored locally at the user device 101, the user device 101 does not necessarily need to be connected to the network 102 during use of the digital therapy. Such an implementation may also be advantageous for users without ready access to a network.

Alternatively, to reduce the data footprint on the user device 101, the digital therapeutic system may be configured to provide the digital therapy 104 via a web-based interface during use. According to one example, the digital therapeutic is provided via an interactive online-based program, which can be accessed via the user device using an up-to-date browser (Google Chrome, Mozilla Firefox, Microsoft Edge, or Safari). In such embodiments, the content rendered to the patient may be generated at the server 103 and provided to the user device 101 via the network 102 in real time. Data transmitted over the network 102 between the user device 101 and the server 103 may be encrypted or otherwise sent using secure communication methods. Similarly, patient data stored at the server may be encrypted while stored at the server 103 and decrypted by the therapy application at the user device 101.

The network 102 may be provided by a wired and/or wireless network. The network 102 may be provided by the internet, another wide area network or a local area network. The therapy application may be distributed to the user device 101 by providing a download from the server 103 via the network 102. The server 103 may be operated by an HCP or wholly independently from the HCP. The server 104 of the digital therapeutic system 100 may be configured to allow the HCP to interact with the patient and/or the therapy application. For example, the server may be configured to allow the HCP to view a status or information associated with the patient (e.g., information related to whether or when the patient has completed one or more modules associated with the therapy application), communicate with the patient, and so on. In such examples, data from the program may only be shared with the HCP if the patient chooses to show the HCP the completed modules using, for example, a patient dashboard.

FIG. 1b illustrates a schematic block diagram of a user device for implementing the digital therapy. The user device 110 comprises one or more processors 112 in communication with memory 114. The memory 114 is an example of a computer readable storage medium. The one or more processors 112 are also in communication with one or more input devices 116 and one or more output devices 118. The various components of the user device 110 may be implemented using generic means for computing known in the art. For example, the input devices 116 may comprise a keyboard or mouse and the output devices 118 may comprise a monitor or display, and an audio output device such as a speaker. In addition, the user device 110 comprises network access circuitry, such as a modem or network adaptor, to provide access to the network to obtain the software application and data. Alternatively, a different data entry means, such as a portable disc drive or data interface may be provided in addition to, or instead of, the network adaptor 120 to provide the therapeutic application and data to the user device 110.

It will be appreciated that, in some examples, the various hardware units may be integrated with one another. For example, where the user device 110 is provided by a tablet computer or cellular telephone, a display of the device 110 may provide both the display of the output device 118 and a touch sensor providing the input device 116.

FIG. 1c illustrates a schematic block diagram of functional units of a digital therapeutic 121. The digital therapeutic 121 is based around a decision engine 122. Ultimately, the decision engine 122 is configured to receive user input 123 and provide user output 124. The user output is provided based on the user input 123 in combination with digital therapeutic data 126 and patient record data 128. The patient record data 128 comprises previously received user input 123 associated with the same patient. The patient record data 128 may also be exported as record of the patient's interaction with the digital therapeutic. The record may include, for example, a summary of patient responses to dialogue.

The digital therapeutic is configured to deliver tailored digital cognitive behavioural therapy (dCBT) for the treatment and management of symptoms and anxiety relating to an ILD, such as IPF, in which future dialogue presented to the user may depend upon the user response to earlier dialogue. The digital therapeutic data 126 comprises content separated into a number of modules. The content within the modules defines what is presented to the user. The content may be formulated to use motivational interviewing techniques in an effort to effectively engage the user by addressing ambivalence through custom-tailored individualization and personalization via the delivery of the simulated therapeutic dialogue. The content may also incorporate principles and techniques from acceptance and commitment therapy, or other evidence-based behaviour change techniques drawn from cognitive behavioural therapy or mindfulness.

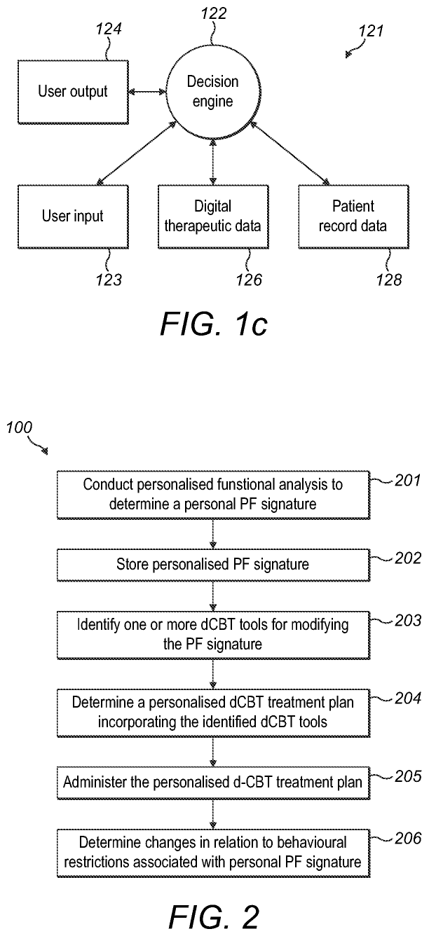

FIG. 2 illustrates exemplary steps (200) for providing a digital therapeutic to a patient diagnosed with an ILD, such as IPF. The process steps illustrated in FIG. 1 are in the form of computer executable instructions, executed on any suitable computing device.

In a first step (201), a personalised functional analysis is conducted with the to determine a personal PF signature. The personal pulmonary fibrosis signature includes one or more anxiety triggers, which the patient identifies as exacerbating the mental suffering of living with an ILD, such as IPF, and thereby causing behavioural restrictions. Behavioural restrictions are any restriction on the behaviour of the patient as a result of an ILD. A patient concerned about an anxiety trigger is likely to avoid situations where such an anxiety trigger may be encountered, thereby restricting the patient's behaviour. Where the anxiety trigger is a fear of taking the prescribed medication due, for example, to concerns over gastro-intestinal side-effects, this will seriously affect patient initiation and adherence to the pharmaceutical element of the treatment regimen.

For example, a patient wanting to perform physical exercise may be anxious that performing physical exercise could exacerbate PF symptoms or even accelerate the progression of the underlying disease. This anxiety could limit or even entirely prevent the patient from performing the desired physical exercise. Such a patient would therefore have a behavioural restriction relating to physical exercise, caused by anxiety relating to an ILD. In this case, the anxiety is in relation to the possible effects of performing physical exercise on the patient's health.

As discussed in more detail below, a method of treatment according to the present disclosure may include repeatedly and systematically exposing the patient to one or more anxiety triggers. The one or more anxiety triggers may be included in one or more situations identified by the patient as including the anxiety triggers. By repeatedly and systematically exposing the patient to these situations, and the anxiety triggers that they include, the patient is taught that the anxiety triggers should not be feared, thereby improving the QoL of the patient.

In the first step (201), the level of anxiety in the patient is determined in relation to one or more of the identified anxiety triggers. A questionnaire may be used to assess the anxiety level of a patient, such as the Generalized Anxiety Disorder 7 item scale (GAD-7).

In a second step (202), the determined PF signature is stored. The stored PF signature includes at least one anxiety trigger which the patient identifies as exacerbating the symptoms of an ILD, thereby causing behavioural restrictions. The stored PF signature may also include additional patient information, such as age, gender, diagnosis (for example IPF), disease progression, interests, fears, and anxiety level.

In a third step (203), one or more digital cognitive behavioural therapy (dCBT) tools are identified for modifying the personal pulmonary fibrosis signature. The identified dCBT tools may be, for example, one or more of: strategies for behaviour change, coping skills to help the patient handle difficult thoughts and feelings, guided sound meditation, a logging flow to gain a better understanding of one's feelings, a step-by-step guide to practice a psychological tool or a text summary of a psychological tool.

In a fourth step (204), a personalised dCBT treatment plan is developed. The dCBT treatment plan includes the one or more dCBT tools that are identified in the third step (203). The determined treatment plan may be configured to be undertaken by the patient over a pre-determined period of time, for example over a period of 8-weeks. The determined dCBT treatment plan may be configured with predetermined daily usage targets, for example with an intended daily use of 10 minutes.

In a fifth step (205), the personalised dCBT treatment plan determined in the fourth step (204) is administered to the patient. Administering the personalised dCBT treatment plan involves the patient making use of the one or more dCBT tools identified in the third step (203). The dCBT treatment plan determined in the fourth step (204) may be delivered in a sequence of modules of neurobehavioral interventions, patient education, and skill-building. The modules may comprise, for example, sound exercises, video exercises, and interactive exercises, among many others, as part of the dCBT tools identified in the third step (203).

In a sixth step (206), subsequent to at least part of the personalised dCBT plan having been administered, changes in relation to the behavioural restrictions associated with the personal PF signature of the patient are determined. Changes may be indicated by comparing the level of anxiety in the patient in relation to the same anxiety triggers identified in the first step (201), the anxiety level of the patient being determined using the same method as was used in the first step (201).

In an example, the personal PF signature of a patient determined in the first step (201) and stored in the second step (202) contains a determined anxiety level in relation to an anxiety trigger that causes behavioural restrictions. The determined anxiety level may be, for example, a value determined using the GAD-7 scale. Alternatively, or additionally, the anxiety level may be self-determined by the patient or may be determined through answers to questions presented to the patient by the digital therapeutic. In the sixth step (206), the anxiety of the patient in relation to the same anxiety trigger action is determined. The anxiety level may be determined by any suitable means or may be self-determined by the patient. The anxiety level determined in the sixth step (206) is compared to the anxiety level that was determined in the first step (201) and is stored in the personal PF signature, thereby to determine whether a reduction in the level of anxiety in relation to the anxiety trigger has been achieved. A reduction in the level of anxiety in relation to the anxiety trigger should indicate a reduction in the associated behavioural restrictions. Any change in the anxiety level of the patient in relation to the one or more anxiety triggers is used to update the personal PF signature of the patient.

Following the assessment performed in the sixth step (206), any or all of the earlier steps (201-205) may be repeated by the patient. As discussed previously, the sixth step (206) may result in a modification to the personal PF signature. For example, any changes in the anxiety level of the patient in relation to one or more anxiety triggers is recorded and will result in a change to the personal PF signature of the patient. When repeating the earlier steps, this modified PF signature may result in different dCBT tools being identified in the third step (203), to further modify the personal PF signature. This would, in turn, result in a different dCBT treatment plan being determined in the fourth step (204) and administered in the fifth step (205). A patient may repeat the entire process (201-206) multiple times to modify different aspects of the PF signature of the patient, thereby.

In some examples, the method of treatment may involve acceptance and commitment therapy (ACT). ACT aims to help the patient to accept their diagnosis and condition (whether the progression of fibrosis is inhibited, or not), and through this acceptance and committed action, to enjoy a more fulfilling life. The use of ACT in this manner thereby improves the QoL of the patient.

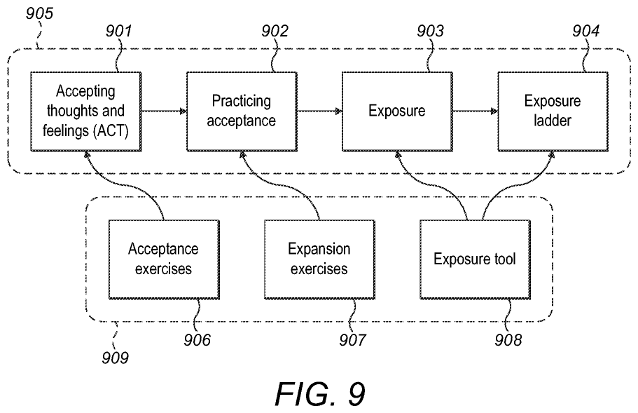

An example of how ACT may be used, as part of the treatment designed to improve the patient's QoL, is illustrated in FIG. 9. In FIG. 9, example treatment steps 905 are shown, with example tools and techniques 909 that may be provided to the patient to assist with the treatment steps 905. As with all treatment steps discussed in this disclosure, treatment steps can be provided via a digital therapeutic according to the present disclosure.

In a first step 901, the patient is helped with accepting feelings and thoughts, through the use of ACT. In this, they can be assisted by a tool configured to provide acceptance exercises 906. The tool can be delivered, for example, by a digital therapeutic according to the present disclosure.

In a second step 902, the patient is directed to practice the acceptance taught in the previous step. This step may involve, for example, a guided sound exercise in which the patient practices opening up to physical sensations and allowing feelings to occur without judgement or avoidance. In this, they may be assisted by a tool configured to provide expansion exercises 907. The tool may provide sound exercises, as discussed previously, and may also involve acceptance exercises. The tool cay be delivered, for example, by a digital therapeutic according to the present disclosure.

In a third step 903, the patient is encouraged to expose themselves to one or more anxiety triggers. In some examples, exposure (and the use of an exposure ladder, as discussed in more detail below) can be undertaken with or without the other ACT steps. The one or more anxiety triggers may be considered in isolation or may be included in a situation identified by the patient as including one or more anxiety triggers. Exposure is performed systematically and repeatedly, with the patient being exposed to one or more anxiety triggers that have been identified by the patient as being feared, avoided or endured with dread.

The goal of exposure is not to reduce or take away anxiety, although this may occur as a side effect of the process. Instead, the goal of exposure is to teach the patient that the identified anxiety triggers are not dangerous, and so should not be feared. By repeatedly and systematically exposing the patient to the identified one or more anxiety triggers in a controlled manner, the patient becomes less afraid of the one or more anxiety triggers, thereby improving the QoL of the patient.

Exposure may involve exposing the patient to any number of anxiety triggers, although in some examples the patient may be encouraged to face only a single anxiety trigger, or a small number of anxiety triggers, in each exposure session. Any anxiety trigger may be used, including those discussed throughout this disclosure.

In some examples, a situation involving one or more anxiety triggers may be identified by the patient. Some examples of situations that may be identified by the patient are: physical activity, discomfort in crowds, discomfort of public transport, thinking about PF, discomfort of healthcare visits, thoughts about the future, social situations, doing things away from home, being outdoors, performing household work and chores, accepting discomfort.

In some examples, the patient is prompted to undertake exposure by choosing a situation involving one or more anxiety triggers and exposing themselves to the situation. In some examples, the patient is prompted to plan the exposure in advance. Planning may involve prompting the patient to identify, in relation to the situation, one or more of: fears, hopes, safety behaviours to drop, and coping strategies to employ during exposure to the situation. In some examples, the fears, hopes, safety behaviours and/or coping strategies may have previously been discussed with the patient through the use of other method steps discussed in this disclosure. For example, through use of the self-directed digital therapeutic program, the patient may have been provided with one or more coping strategies to employ. The patient may also be prompted to identify, ahead of the exposure, their perceived anxiety in relation to the exposure.

After the patient has undertaken the exposure, the patient may be prompted to provide feedback in relation to the exposure. For example, the patient may be prompted to provide a difficulty score associated with the exposure. The patient may also be prompted to indicate to what extent their fears ahead of the exposure came true. Additional feedback regarding the use of coping strategies and safety behaviours may also be prompted.

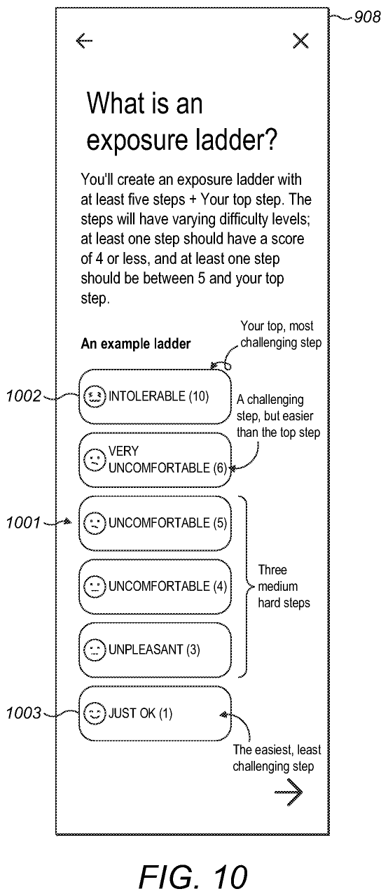

In some examples, exposure may be managed through the use of an exposure ladder 904. In some examples, exposure and/or the use of an exposure ladder can be assisted through the use of an exposure tool 908 delivered, for example, by a digital therapeutic according to the present disclosure. FIG. 10 illustrates an example of an exposure ladder 1001 provided by a the digital therapeutic according to the present disclosure. In the illustrated example, the exposure ladder is provided using an exposure tool 908.

The exposure ladder 1001 allows the patient to identify for a given situation, a variety of possible steps that each include one or more anxiety triggers. For example, if the identified situation is physical exercise, different steps could involve going for a short walk, going for a longer walk, attending a dance class, and doing a workout at a gym. Each of these steps involves one or more anxiety triggers and for each step the patient is prompted to select a level of anxiety that they feel for undertaking this step. The identified steps are then ordered by the level of anxiety identified by the patient, to form an exposure ladder for the identified situation. In the example illustrated in FIG. 10, the steps are ordered by the level of anxiety identified by the patient, with the most challenging step 1002 at the top of the ladder, and the least challenging step 1003 at the bottom of the ladder.

In an alternative example, the exposure ladder may additionally, or alternatively, be formed in relation to anxiety triggers identified in isolation, rather than as part of a particular situation.

Exposure using the exposure ladder is undertaken by prompting the patient to plan an exposure for one of the steps on the ladder. Typically, the step with the lowest level of anxiety identified by the patient is chosen first. Exposure is then undertaken, and the patient is then prompted to provide feedback regarding the exposure. Exposure may be undertaken for a given step multiple times.

As discussed previously, the aim of exposure is not to reduce or take away anxiety, although this may occur as a side effect of the process. Instead, the goal of exposure is to teach the patient that the identified anxiety triggers are not dangerous, and so should not be feared. In examples where the exposure ladder is used, the patient will slowly progress up the ladder undertaking exposures to steps with a higher perceived level of anxiety. As exposure to each step is undertaken (perhaps multiple times), the patient is taught that the anxiety triggers associated with each step are not dangerous and should not be feared.

By repeatedly and systematically exposing the patient to the identified one or more anxiety triggers in a controlled manner, for example by use of any of the above-described techniques, the patient becomes less afraid of the one or more anxiety triggers. This in turn reduces behavioural restrictions associated with the personal PF signature of the patient, thereby improving the QoL of the patient.

Further anxiety triggers could include, but are not limited to, taking medication, going outside, having to carry and/or use equipment to assist breathing such as an oxygen tank, entering social environments, meeting with people, coughing, shortness of breath, safety of activities, and perceived risk of catching an infection while doing an activity.

Anxiety caused by taking medication is due to two main triggers. The two leading anti-fibrotic medications on the market are known to cause nausea, vomiting, diarrhoea, and abdominal distress. The awareness of these adverse side-effects is well known in the pulmonary fibrosis community, so much so that many do not ever initiate medication with either of these two anti-fibrotics. Not every patient experiences these side-effects, and not everyone who has these side-effects stops taking the medication. Nevertheless, the percentage of people who initiate this potentially life-extending medication is only around 27% in the US.

Another problem with medication anxiety is that the gastro-intestinal side-effects can lead to fear of leaving the house, social isolation, and reduction of activity and socialisation which has negative effects on the disease progression.

The digital therapeutic addresses the anxiety triggered by the idea of taking medication through dCBT treatment modules configured to address fear triggers and aversion issues, gradually introducing concepts that encourage at least trying, and getting first-hand information. Using dCBT modules, coping mechanisms are introduced, anchored, and repeated, including knowledge of how various medications works, potential benefits, patient story videos of people who did take medicines for PF and saw a benefit, nutrition assistance, mitigating side effects of medications with timing of meals. The dCBT incorporates treatment modules for overcoming fear of possible nausea, vomiting, and diarrhea, and coping with it if it does occur.

In some embodiments, dCBT treatment modules are provided which address the fear of trying new medicines in clinical trials. The only way to get new treatments that save lives is to have enough subjects in clinical trial to test them when they get to the appropriate phase.

The combination of these mechanisms of action, building toward an exposure and tools to control these side-effects and fear of side-effects, leads to increased initiation of medicine protocols and subsequently better adherence to lung fibrosis drug treatment protocols.

Adherence to the pharmaceutical treatment regimen can be measured by patient-reported outcome measures captured by the digital therapeutic program, physician reporting for the patient within the healthcare system, inference through patient interactions with the digital therapeutic program, and/or progression of the ILD as determined, for example, using forced vital capacity (FVC) measurements.

In some examples, an additional electronic device may be used to measure and record patient health data during the digital therapeutic treatment. For example, a smart watch may be used to measure and record information such as the heart rate of the patient during treatment. Such health data can be used by the digital therapeutic to estimate the anxiety level of the patient during treatment. This additional estimate of the patient anxiety level may be used in combination with the information provided by the patient via anxiety assessments performed during treatment (for example, through use of the GAD-7 scale) to estimate the anxiety level of the patient more frequently. In some examples, this may identify further anxiety triggers, or to highlight known anxiety triggers that are particularly strong. In some examples, the additional electronic device may measure and record further information such as respiration rate, heart rate variability (HRV), steps per day, oxygenation.

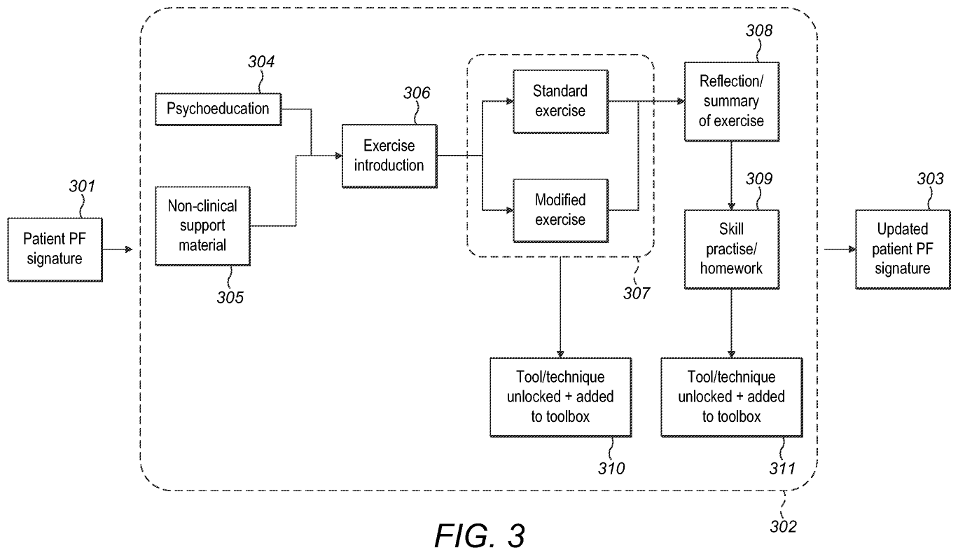

FIG. 3 illustrates an example of a patient pathway using a digital therapeutic according to the present disclosure. In particular, FIG. 3 illustrates an example of the treatment path that may be taken by a patient while progressing through the third, fourth, fifth and sixth steps (203-206) discussed above. In the treatment path illustrated in FIG. 3, the patient begins with a personal PF signature (301), and after a personalised dCBT treatment plan has been administered (302), the personal PF signature is modified (303). The patient pathway illustrated in FIG. 3 may be undertaken multiple times, with the personal PF signature being modified each time. Each time the patient pathway is undertaken, a different personalised dCBT treatment plan may be used.

The personalised dCBT treatment plan illustrated in FIG. 3 begins with the patient being provided with psychoeducation (304) and/or non-clinical support material (305). This may provide helpful background information to the patient to help support them through the treatment plan.

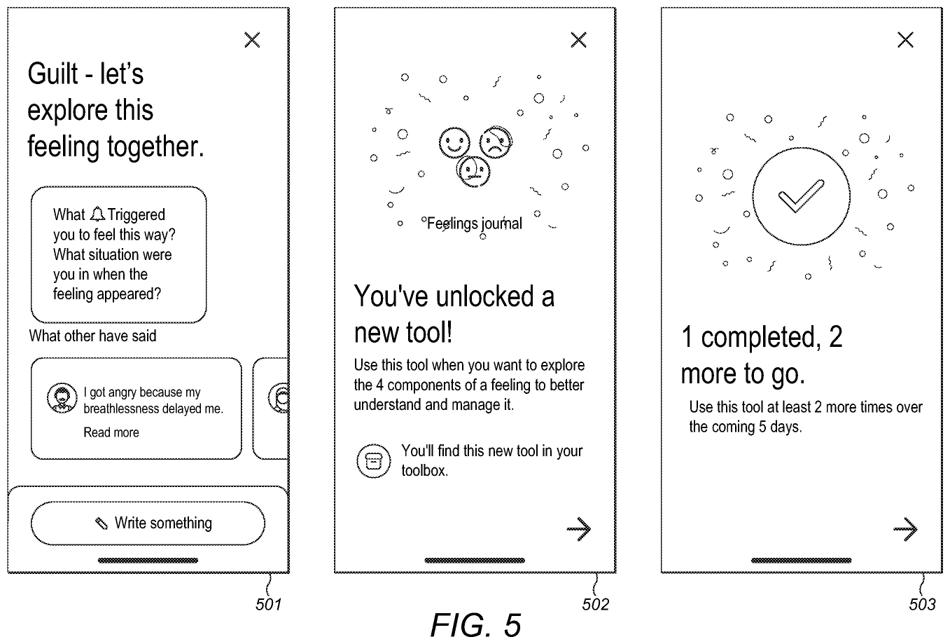

The patient is then introduced (306) to one or more exercises, which may be presented in the form of modules (as discussed in more detail below). The exercise introductions (306) may include questions to help modify the exercise to match the needs of the patient more closely. An example of how such an introduction may be presented is illustrated in the first screen (501) of FIG. 5, which is discussed in more detail below.

Following the exercise introduction (306), the patient is presented with an exercise to perform (307). The exercise (307) may be a standard exercise, or it may be an exercise that has been modified in order to match the personalised requirements of the patient more closely. After completion of the exercise (307), a “tool” may be unlocked and added to the patient “toolbox” (310). An example of this process is illustrated in the second screen (502) of FIG. 5 and is discussed in more detail below. “Tools” and the patient “toolbox” are also discussed in more detail below.

After completion of the exercise (307), the patient is then presented (308) with a summary of the exercise in order to reflect on the exercise and the progress that may have been made while performing the exercise. The patient is then given “homework” (309) which involves practising the exercise in order for the exercise to provide further benefits to the patient. The patient may be instructed to repeat the skill practise a number of times (the number being pre-set or may be determined by reference to the personal PF signature of the patient). An example of this process is illustrated in the third screen (503) of FIG. 5 and is discussed in more detail below. Following completion of the skill practise, a tool may be unlocked and added to the patient toolbox (311).

After completion of the administered personalised dCBT treatment plan (302), the personal PF signature of the patient may be updated (303).

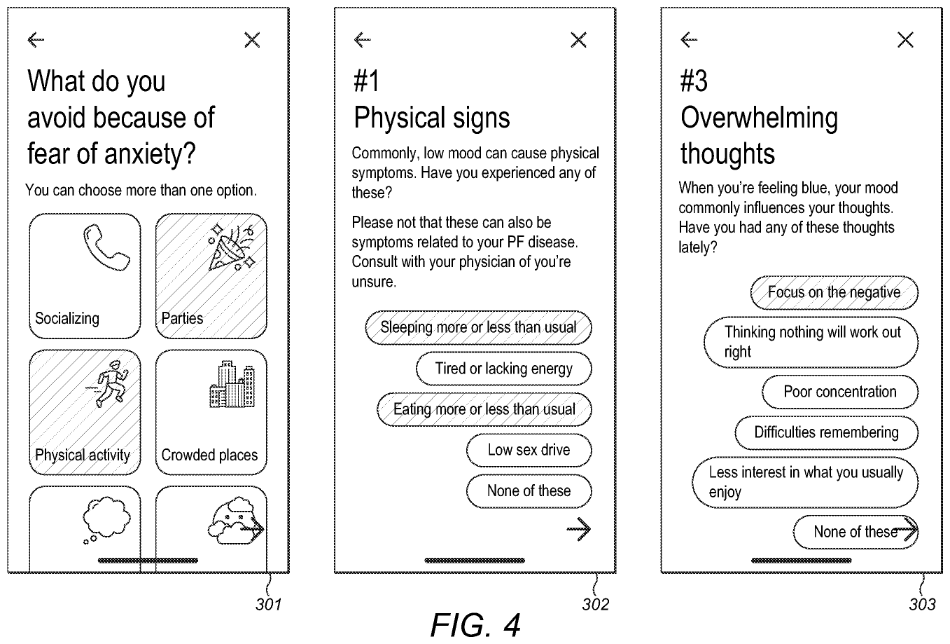

FIG. 4 illustrates examples of how parts of the personalised functional analysis may be performed in the first step (201) to determine a personal PF signature that is stored in the second step (202). The examples in FIG. 4 illustrate an example implemented in an app for use on an electronic device such as a smart phone. The illustrations (301, 302, 303) in FIG. 3 show examples of how various part of the personalised functional analysis may be performed to determine and store the personal PF signature of the patient.

The first screen (301) of FIG. 4 shows the app asking the patient to identify activities that they avoid due to fear of anxiety. The activities identified by the patient are anxiety triggers that exacerbate the symptoms of an ILD, such as IPF. By avoiding these activities, the patient is self-imposing behavioural limitations to avoid the identified anxiety triggers. The second screen (302) shows the app asking the patient to identify any physical symptoms they may be experiencing. The third screen (303) shows the app asking the patient to identify any overwhelming thoughts they may be experiencing. The answers that the patient inputs into each of these screens (302, 302, 303) are stored in the personal PF signature of the patient.

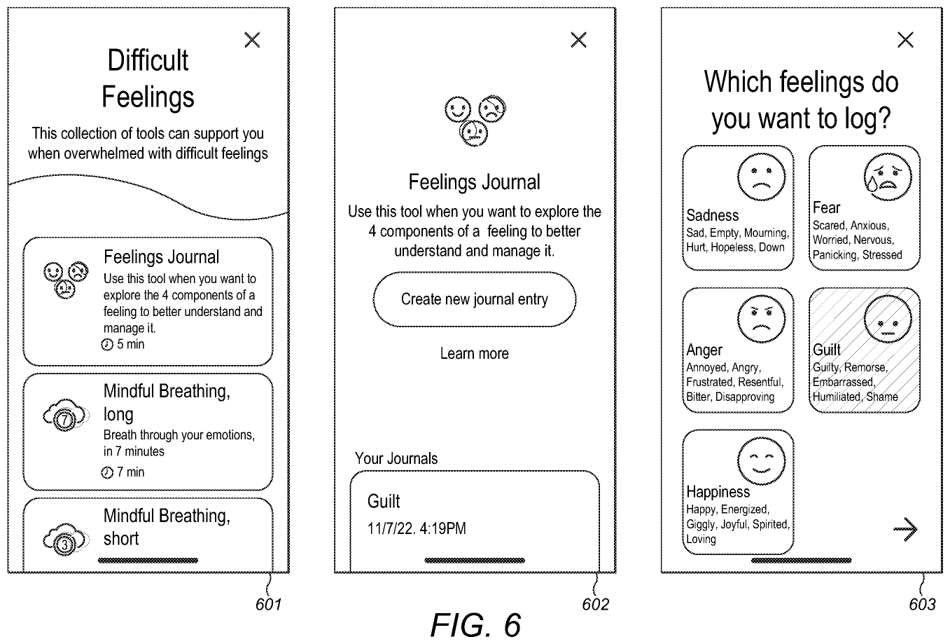

FIGS. 5 and 6 illustrate examples of parts of a process of identifying one or more dCBT tools for modifying the personal PF signature of the patient and for determining a personalised dCBT treatment plan incorporating the identified tools, as is performed in the third and fourth steps (203, 204) discussed above. FIG. 5 illustrates an example of how a patient is introduced to a new tool that may be used as part of a dCBT treatment plan to modify the personal PF signature of the patient. The first screen (501) illustrates an example of how a tool may be introduced to a patient as a potential tool for modifying the personal PF signature of the patient. The second screen (502) shows the completion of an introduction to the tool and shows that the tool has been added to the patient toolbox. The third screen (503) shows instructions given to the patient to repeat usage of the tool at least two more times over a period of five days.

The first screen (601) of FIG. 6 illustrates a selection of tools presented to a patient to help modify the personal PF signature of the patient. This may be an example of a personalised dCBT treatment plan. The second (602) and third (603) screens of FIG. 6 illustrate a tool that is part of the personalised dCBT treatment plan. The example illustrated in the second (602) and third (603) screens is a “feelings journal” that can be used by the patient to record feelings so that the feelings may be explored through use of the tools available to the patient.

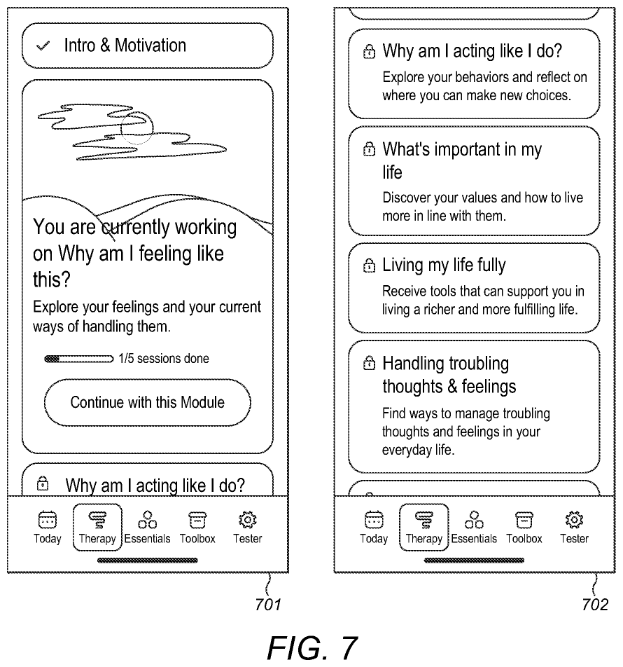

FIG. 7 illustrates examples of part of a process for administering the personalised dCBT treatment plan, as is performed in the fifth step (205) discussed above. The first screen (701) of FIG. 7 illustrates an example of a therapy module that the patient is part way through completing (one out of the prescribed five sessions is shown as having been completed). The second screen (702) shows other modules that are available for the patient to use as part of the personalised dCBT treatment plan.

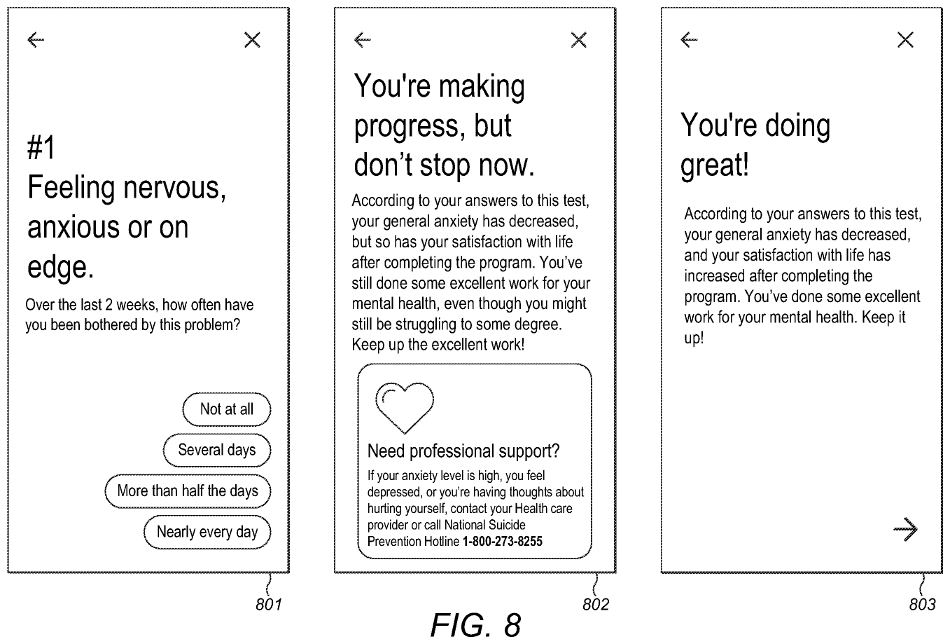

FIG. 8 illustrates examples of part of a process for determining changes in relation to the behavioural restrictions associated with the personal PF signature of the patient, as performed in the sixth step (206) discussed above. The first screen (801) of FIG. 8 illustrates an example of a question that may be presented to a patient following completion of a personalised dCBT treatment plan, in order to determine changes to the personal PF signature. The second screen (802) illustrates information that may be presented to the patient if the determined changes to the personal PF signature are only somewhat positive. In the example illustrated in the second screen (802), the anxiety levels of the patient have been reduced, but the patient has indicated that their quality of life has also decreased. The patient is encouraged to focus on the fact that their anxiety levels have reduced, despite the fact that they may not be entirely satisfied with other aspects of their life. The patient is also presented with information to contact a HCP if needed. The third screen (803) illustrates information that may be presented to a patient if the determined changes to the personal PF signature are largely positive. In this example, the patient has indicated that their anxiety levels have reduced and their satisfaction with their life has also increased.

FIG. 3 illustrates an example of a typical patient journey of a patient making use of a digital therapeutic according to the present disclosure. The first step of a patient journey is the confirmation of the ILD diagnosis (201). As discussed in more detail in the background section, an ILD diagnosis can be very difficult for a patient to cope with mentally. ILDs such as IPF often have no identifiable cause, and few treatment options beyond simply managing the day-to-day symptoms of the disease. The impact on the patient's mental health can be particularly exacerbated by a deterioration in physical symptoms, which can heavily impact upon the daily life of the patient.

Following diagnosis, the digital therapeutic disclosed herein may be made available to the patient. In a preferred example, the digital therapeutic is arranged into several modules (210). In a preferred example, the modules (210) are separated into two groups: “information modules” (211), and “therapy modules” (212). The modules are provided to the patient via an electronic device, for example through an app running on a smartphone.

The information modules (211) are non-interventional PF-specific information and activities modules. The information modules (211) may include general wellness modules and may provide relevant information about ILDs such as IPF, tips on nutrition specific to ILDs and intestinal issues that occur with some treatments, PF-specific breathing exercises for coping with shortness-of-breath, suggestions on better sleep habits and ways to incorporate an acceptable level physical activity. The information modules (211) may help to support patients in creating a sustainable and healthy lifestyle with an ILD and helping to facilitate engagement with the therapy modules (212).

Examples of possible information modules (211) are:
“Coping with PF”: Introducing breathing exercises and energy conservation strategies to support with PF-symptoms like coughing and shortness of breath.
“Caring for Myself”: Information on good eating and sleep habits for PF-patients, as well as how to work with self-compassion. Support to create a good sleep routine.
“Physical Activity”: Information and support to create a sustainable habit of physical activity.

The therapy modules (212) are interventional CBT modules. In some examples, each therapy module (212) is designed to take the patient approximately 8-weeks to work through, if used as intended.

Examples of possible therapy modules (212) include:
“Why am I feeling like this?”: Psychoeducation about emotions, anxiety, and low mood. The module helps the patient explore their feelings towards their ILD and helps with mapping current strategies to handle difficult emotions.
“Why am I acting like I do?”: Mapping patient's behaviours relating to the emotional aspects of ILDs and their short- and long-term consequences. This is called Functional Analysis and is the basis of CBT treatment. Introduction of the choice point, which is an extension of functional analysis and is a core concept of modern Acceptance and Commitment Therapy (ACT).
“What's important in my life?”: Exploring the patient's Values, how the patient wants to live their life and how ILD restricts them.
“Living my life fully”: Working with “Committed Action” to live more in line with values. Mapping and finding strategies to overcome the obstacles to live in accordance with values. Working with Exposure to difficult feelings and thoughts, an important intervention part of a range of CBT treatments.
“Handling troubling thoughts and feelings”: Introducing and practicing the psychological strategies of Acceptance, Defusion and Mindfulness as tools to cope with difficult thoughts and feelings.
“Sustainable change”: Create strategies for the patient to work with the tools they have received going forward. This type of maintenance plan is often the last module in all forms of CBT treatments.

As discussed previously, the first step (201) in using the digital therapeutic involves conducting a personalised functional analysis with the patient to determine a personal PF signature. In some examples, this step may involve assessing the patient anxiety levels, for example by using a GAD-7 scale. Patient anxiety levels can also be determined by answering questions presented by the digital therapeutic. Additionally, or alternatively, the patient anxiety level can simply be self-determined by the patient, for example by choosing an anxiety level on a pre-determined scale provided by the digital therapeutic.

In a preferred example, an anxiety level screening (202) is performed before therapy modules (212) are made available to the patient. This anxiety level screening may be separate to the assessment of anxiety performed in the first step (101) discussed above. The anxiety level screening may be, for example, in relation to generalised feelings instead of in relation to a particular anxiety trigger. Alternatively, the anxiety level screening (202) may be in relation to the one or more anxiety triggers causing behavioural limitations identified by the patient in the first step (101).

The anxiety level screening (202) may be performed using the GAD-7 scale. Patients can determine their need for psychological support by answering the GAD-7 questions within the digital therapeutic app. This assessment is not considered to be a diagnosis, but instead is a way for the patient themselves to gain an understanding of the relative status of their mental health. If the score is above a certain level on the GAD-7 (5 or higher on a scale of 0-21), patients may be referred to consult with an HCP (for example an ILD specialist or a primary care physician).

Self-assessments may also be provided mid-way through the determined treatment program as well as after completing treatment. The self-assessments may be in relation to general anxiety. The self-assessments may alternatively, or additionally, be in relation to the one or more anxiety triggers identified in the first step (101) and stored in the personal PF signature of the patient. The self-assessments are designed to be a way for the patient themselves to get an understanding of their progress. In some examples, the self-assessment results may not be shared with healthcare providers automatically, although patients may choose to share their results verbally or by sharing a summary (for example, a final PDF form) provided by the digital therapeutic.

In a preferred example, if the anxiety level screening (202) determines that the anxiety level of the patient is below a threshold value (204), the patient is only given access to the information modules (211). In examples where the GAD-7 score is used, a patient may be determined to be below the threshold anxiety level (204) if they receive a score equal to or less than 4 on the GAD-7 scale.

If the anxiety level screening (202) indicates that the patient has an anxiety level above a threshold level (203), the patient may be provided access to the therapy modules (212). In examples where the GAD-7 score is used, a patient may be determined to be above the threshold anxiety level (203) if they receive a score equal to or greater than 5 on the GAD-7 scale.

In some examples, if the patient is determined to have an anxiety level above the threshold level (203), the patient may be referred to an HCP before access is given to the therapy modules (212). In these examples, the HCP may determine which of the therapy modules (212) may be appropriate for the patient to use. In some examples, access to the therapy modules (212) is only given to the patient if the therapy module (212) is prescribed to the patient by the HCP. In such examples, each therapy module (212) may be unlockable by means of a code which is provided to the patient following prescription of the module by the HCP.

In a preferred example, the modules (211, 212) may comprise “Sessions”, “Tools” or “Tool Practice”.

“Sessions” are the primary scheduled interaction between the patient and the treatment, focusing on psychoeducation through text, video and audio, all of which introduce psychological concepts and strategies from CBT and ACT. The Sessions may also include stories from other ILD patients and interactive exercises where the patient is encouraged to reflect upon their own situation by answering questions. Sessions may be built to resemble face-to-face therapy sessions with a psychologist in structure and content, although they are entirely software-based and no actual psychologist is involved.

Strategies for behaviour change, as well as coping skills to handle difficult thoughts and feelings, are referred to as “Tools” in the modules and in the digital therapeutic. As the patient progresses through Sessions, new Tools are unlocked and added to the patient's Toolbox. Tools may be, for example, a guided sound meditation, a logging flow to allow the patient to gain a better understanding of their feelings, a step-by-step guide to practice a psychological tool or a text summary of a psychological tool. The Tools are designed to resemble the tools that a patient would be introduced to in face-to-face therapy with a psychologist.

“Tool Practice” is the skill building activity of using and practicing the Tools in everyday life. The patients may be tasked to use a Tool for a set number of times during a set number of days. Once the task is completed, the patient may be asked to reflect upon what they have done before proceeding to the next task. Tool Practice resembles “homework” that is an established practice in face-to-face CBT. In traditional CBT, patients would receive homework to complete during the week in-between therapy sessions. A further advantage of the digital therapeutic disclosed herein is that this practise work may be scheduled at the patient's own pace.

A digital therapeutic app designed according to the present disclosure has been tested in a pilot study involving 10 patients. For admittance to the pilot study, the following inclusion criteria were imposed on potential trial patients:
1. Age ≥22 years at the time of signing the informed consent;
2. Self-reported Diagnosis of Idiopathic Pulmonary Fibrosis;
3. A 7-item generalized anxiety questionnaire score of ≥5 at baseline;
4. If currently on prescribed medication for depression/anxiety, a stable dose for at least 4 weeks prior to enrolment; and,
5. Capable of using a mobile device and common applications and has an appropriate mobile or tablet device.

The following exclusion criteria were used:
1. Manic disorders, psychotic disorders, suicidal ideation, schizophrenia, self-harm, or alcohol/drug abuse during the past 6 months prior to baseline;
2. Cognitive impairment e.g., dementia preventing engagement with dCBT;
3. Verbal and/or written communication problems limiting ability to engage with dCBT;
4. Inability to comply with investigation procedures, due to severe medical conditions or otherwise;
5. Life expectancy of less than 6 months as determined by the need for palliative care; and,
6. Currently receiving cognitive behavioural therapy.

The results of the pilot study are illustrated below in Table 1. The GAD-7 scores were self-reported by patients, with a score taken at the start and at the end of the study on a 0-21 point scale. On a GAD-7 scoring chart, the determined anxiety of the patient is as follows: 0-5 mild, 6-10 moderate, 11-15 moderately severe, 15-21 severe anxiety.

As noted above, the patient had to register a score of at least 5 to be admitted to the pilot study. Between the start and end scores, the patient used the digital therapeutic app to provide dCBT in accordance with the present disclosure.

TABLE 1

| | Start GAD-7 | End GAD-7 | Change in | Percentage |
|---|---|---|---|---|
| Patient no. | score | score | GAD-7 score | change |
| 1 | 5 | 1 | 4 | 80% |
| 2 | 8 | 2 | 6 | 75% |
| 3 | 10 | 2 | 8 | 80% |
| 4 | 8 | 3 | 5 | 62.5% |
| 5 | 5 | 2 | 3 | 60% |
| 6 | 12 | 7 | 5 | 42% |
| 7 | 14 | 7 | 7 | 50% |
| 8 | 9 | 7 | 2 | 22% |
| 9* | 7 | 6 | 1 | 14% |
| 10* | 15 | 14 | 1 | 7% |
| | | Average | 4.2 | 49.25% |
| | | Average | 5 | 59% |
| | | (1 to 8 only) | | |

As illustrated in Table 1, all patients who used the digital therapeutic observed a reduction in their GAD-7 scores, with an average reduction of 4.2 points or 49% of their GAD-7 score. It was noted after the trial that patients 9 and 10 did not use the digital therapeutic app for the full amount of time required during the trial. These results are therefore marked with an asterisk and should not be considered to be entirely representative of the effectiveness of the digital therapeutic app, when used as intended. However, even with incomplete usage, patients 9 and 10 did still observed a small reduction in their anxiety levels following use of the digital therapeutic treatment.

The remaining patients, numbered 1 to 8 in Table 1, observed an average reduction of 5 points in their GAD-7 score following treatment using the digital therapeutic. This represents an average reduction of 59% in the measured anxiety levels following treatment using the digital therapeutic.

Of the ten patients recruited to the study, six were concomitantly treated with antifibrotic drugs (pirfenidone or nintedanib), four were treated with drugs for anxiety (buspirone, clonazepam, desvenlafaxine, fluoxetine, gabapentin and/or lorazepam) and five were treated with proton pump inhibitors (omeprazole, pantoprazole or rabeprazole). Considering that DTx reduced the GAD-7 score in all 10 patients, it can be concluded that the dCBT resulted in reduced anxiety beyond any anxiolytic or other therapeutic effects of these drugs.

A digital therapeutic app designed according to the present disclosure has been tested in a further study involving 108 patients. The study (CT.gov listing: NCT05330312), was a randomized controlled clinical investigation conducted in the US. 108 Adults with pulmonary fibrosis confirmed by a CT scan and anxiety symptoms fulfilling ≥5 points on the Generalized Anxiety Disorder 7-items scale (GAD-7) at baseline were enrolled on the study.

For admittance to this study, the following inclusion criteria were imposed on potential trial patients:
1. Age ≥22 years at the time of signing the informed consent;
2. Diagnosis of Pulmonary Fibrosis;
3. A GAD-7 score of ≥5 at pre-screening and baseline;
4. If currently on prescribed medication for depression/anxiety, a stable dose for at least 4 weeks prior to enrolment;
5. CT scan report within 5 years prior to baseline with signs of PF (interstitial changes); and,
6. Capable of using a mobile device and common applications, and has an appropriate mobile or tablet device.

The following exclusion criteria were used:
1. Self-reported manic disorders, psychotic disorders, suicidal ideation, schizophrenia, self-harm, or alcohol/drug abuse during the past 6 months prior to baseline;
2. Self-reported history of hospital admissions due to suicidal behaviour or attempts, as judged by the investigator or designee;
3. Self-reported previous or current diagnosis of major depressive disorder, as judged by the investigator or designee;
4. Verbal and/or written communication problems limiting ability to engage with dCBT-IPF;
5. Inability to comply with investigation procedures, due to e.g. cognitive impairment or severe medical conditions as judged by the investigator or designee; and,
6. Currently receiving cognitive behavioural therapy.

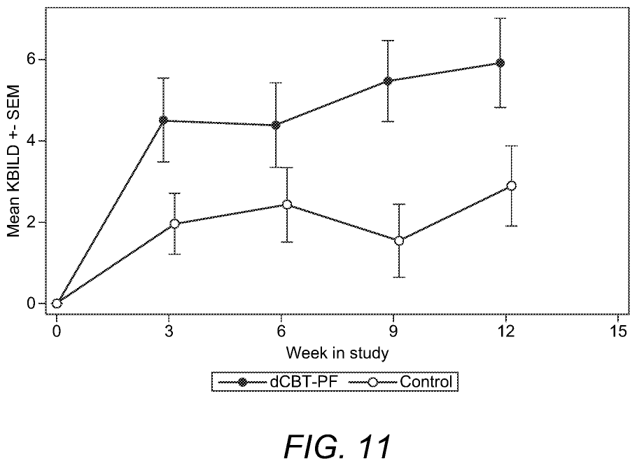

The results of this further study are illustrated in FIG. 11.

Results show that the primary endpoint, change in GAD-7 from baseline to week 9, the group using the digital therapeutic app designed according to the present disclosure had an estimated LS mean change of −2.7 points (95% confidence interval: −4.2 to −1.2, p=0.0006) compared to the control group. This highly significant difference was still seen at week 12, with an LS mean difference of −3.0 points (95% confidence interval: −4.5 to −1.5, p=0.0001).

A treatment effect was also observed on the K-BILD total score, which is a validated quality of life (QoL) measure for interstitial lung disease patients. KBILD results showed the group using the digital therapeutic app designed according to the present disclosure had an estimated improvement at week 9 of 4.4 points (95% confidence interval: 1.6 to 7.1, p=0.0019) compared to the control group. There are three domains of the KBILD, divided into Breathless/Activities, Chest symptoms, and Psychological. The patients using the digital therapeutic app designed according to the present disclosure showed improvement over control in each domain at week 9 and at 12-week follow-up, as illustrated in FIG. 11.

Although the description above is with reference to ILDs such as IPF, the digital therapeutic described herein may in addition be used to treat anxiety caused by any disease or condition with dyspnea/difficulty breathing as a symptom, including asthma, chronic obstructive pulmonary disease, emphysema, pulmonary hypertension such as pulmonary arterial hypertension, respiratory infections such as bronchitis and pneumonia, airway-blocking cancers including lung cancer, cardiac diseases such as heart failure and myocardial ischemia, neuromuscular disorders affecting the respiratory muscles, pregnancy, obesity and/or psychogenic breathing disorders.

The drugs listed below are used in one or more of these other common chronic conditions that can cause anxiety due to dyspnea/difficulties breathing.

Short-acting bronchodilators such as albuterol, ipratropium, metaproterenol, levalbuterol, salbutamol, terbutaline.

Long-acting bronchodilators such as aclidinium, arformoterol, formoterol, indacaterol, tiotropium, salmeterol, umeclidinium, vilanterol, umeclidinium, glycopyrrolate, aclidinium.

Inhaled corticosteroids such as fluticasone, flunisolide, budesonide, mometasone, beclomethasone, ciclesonide, triamcinolone acetonide.

Inhaled drug combinations such as fluticasone+vilanterol, fluticasone+umeclidinium+vilanterol, formoterol+budesonide, salmeterol+fluticasone, aclidinium+formoterol, albuterol+ipratropium, formoterol+glycopyrrolate, glycopyrrolate+indacaterol, mometasone+formoterol, olodaterol+tiotropium, umeclidinium+vilanterol.

Phosphodiesterase-4 inhibitors such as cilomilast, ibudilast, roflumilast.

Leukotriene modifying drugs such as montelukast, zafirlukast, zileuton.

Biologic drugs such as omalizumab, benralizumab, dupilumab, mepolizumab, reslizumab.

Angiotensin-converting enzyme inhibitors such as alacepril, captopril, zofenopril, enalapril, ramipril, quinapril, perindopril, lisinopril, benazepril, imidapril, trandolapril, cilazapril, fosinopril.

Angiotensin II receptor blockers such as eprosartan, valsartan, losartan, azilsartan, olmesartan, telmisartan, fimasartan, irbesartan.

Beta receptor blocking agents such as atenolol, bisoprolol, carvedilol, labetalol, metoprolol, propranolol, sotalol.

Diuretics such as chlorothiazide, chlorthalidone, hydrochlorothiazide, indapamide, metolazone, bumetanide, ethacrynic acid, furosemide, torsemide, amiloride, eplerenone, spironolactone, triamterene.

Positive inotropes such as dopamine, dobutamine, levosimendan, milrinone, digoxin, amrinone, enoximone.

Sodium-glucose co-transporter 2 inhibitors such as canagliflozin, dapagliflozin, empagliflozin, ertugliflozin, ipragliflozin, remogliflozin etabonate, sergliflozin etabonate, sotagliflozin, tofogliflozin.

Other relevant pharmaceutical treatments tadalafil, ambrisentan, bosentan, opsumit, epoprostenol, epoprostenol, treprostinil, iloprost, selexipag, riociguat, ivabradine, hydralazine, isosorbide dinitrate, vericiguat, theophylline, angiotensin-(1-7), omeprazole, esomeprazole, ilaprazole, lansoprazole, dexlansoprazole, pantoprazole, rabeprazole.

1. A method of treating a patient diagnosed with an interstitial lung disease (ILD), comprising:
administering a pharmaceutical drug for treating pulmonary fibrosis in a treatment regimen effective to treat fibrosis, inflammation, and/or vasculopathy; and,
in conjunction with the administration of the pharmaceutical drug, engaging in a self-directed digital therapeutic program that improves the quality of life of the patient, the self-directed digital therapeutic program including steps of:
conducting a personalised functional analysis with the patient to identify one or more anxiety triggers which the patient identifies as causing behavioural restrictions;
determining a personalised treatment plan incorporating progressive exposure of the patient to one or more of the identified anxiety triggers;
administering the personalised treatment plan; and
through progressive exposure to the one or more anxiety triggers, reducing the fear that the patient associates with the one or more anxiety triggers, thereby reducing behavioural restrictions and improving the quality of life of the patient.

2. The method according to claim 1, wherein the pharmaceutical drug for treating pulmonary fibrosis is an angiotensin II type 2 receptor agonist (ATRAG).

3. The method according to claim 2, wherein the ATRAG is N-butyloxycarbonyl-3-(4-imidazol-1-ylmethylphenyl)-5-iso-butylthiophene-2-sulfonamide (Compound 21 or ‘C21’).

4. The method according to claim 1, wherein the pharmaceutical drug for treating pulmonary fibrosis is selected from pirfenidone, nintedanib, treprostinil, pamrevlumab, nalbuphine, thalidomide, phosphodiesterase 4 inhibitor BI 1015550; an anxiolytic such as buspirone, clonazepam, desvenlafaxine, fluoxetine, gabapentin and/or lorazepam; or a proton pump inhibitor such as omeprazole, pantoprazole or rabeprazole.

5. The method according to claim 1, wherein after exposure of the patient to one or more of the identified anxiety triggers, the patient is prompted to provide an assessment of the exposure.

6. The method according to claim 5, wherein the assessment includes a determination of the level of fear that the patient felt in relation to the one or more anxiety triggers during the exposure.

7. The method according to claim 5, wherein the personalised treatment plan is modified according to the assessment of the exposure provided by the patient.

8. The method according to claim 5, wherein the assessment is provided by the patient using a difficulty score.

9. The method according to claim 8, wherein if the difficulty score is above a threshold, the personalised treatment plan is modified to reduce the level of exposure to the one or more anxiety triggers.

10. The method according to claim 8, wherein if the difficulty score is below a threshold, the personalised treatment plan is modified to increase the level of exposure to the one or more anxiety triggers.

11. The method according to claim 1, wherein the personalised treatment plan includes the use of an exposure ladder.

12. A method according to claim 1, wherein the digital therapeutic program comprises the computer implemented steps of:
conducting a personalised functional analysis with the patient to determine a first personal pulmonary fibrosis (PF) signature, the first personal PF signature including one or more anxiety triggers which the patient identifies as leading to possible exacerbations of the symptoms of an ILD and thereby causing behavioural restrictions;
storing the first personal PF signature;
identifying one or more digital cognitive behavioural therapy (dCBT) tools for modifying the personal PF signature;
determining a first personalised dCBT treatment plan which incorporates the use of the identified dCBT tools;
administering the first personalised dCBT treatment plan; and,
determining changes in relation to the behavioural restrictions associated with the first personal PF signature of the patient.

13. The method according to claim 12, further comprising the step of determining the anxiety level of the patient in relation to one or more of the identified one or more anxiety triggers, before administering the first personalised dCBT treatment plan.

14. The method according to claim 12, further comprising the step of determining the anxiety level of the patient in relation to the one or more anxiety triggers after administering the first dCBT treatment plan.

15. The method according to claim 12, further comprising the step of comparing the determined anxiety levels to assess changes in the first personal PF signature of the patient.

16. The method according to claim 12, wherein administering the first personal dCBT treatment plan comprises steps of:
providing the patient with an exercise introduction incorporating one or more questions for the patient to answer, wherein the exercise introduction is related to an exercise that is part of an identified dCBT tool for modifying the first personal PF signature;
in response to the answers provided by the patient, modifying the exercise; and,
providing the patient with the modified exercise.

17. The method according to claim 12, wherein determining changes in relation to the behavioural restrictions associated with the first personal PF signature of the patient further comprises a step of determining changes in the perceived quality of life of the patient.

18. The method according to claim 12, further comprising the steps of:
determining a second personal PF signature, by modifying the first personal PF signature to incorporate the determined changes in relation to the behavioural restrictions associated with the first personal PF signature of the patient; and,
storing the second personal PF signature.

19. The method according to claim 18, further comprising the steps of:
identifying one or more digital cognitive behavioural therapy (dCBT) tools for modifying the second personal PF signature;
determining a second personalised dCBT treatment plan which incorporates the use of the identified dCBT tools;
administering the second personalised dCBT treatment plan; and,
determining changes in relation to the behavioural restrictions associated with the second personal PF signature of the patient.

20. The method according to claim 1, wherein, as a consequence of said anxiety treatment, one or more of the symptoms of the ILD are positively affected.

21. The method according to claim 20, wherein the symptoms include shortness of breath and/or cough.

22. The method according to claim 1, wherein the anxiety treatment leads to an improvement or an at least partial restoration of the patient's lung function/vital capacity.

23. The method according to claim 1, wherein said anxiety treatment leads to a reduction of morbidity and/or mortality associated with the ILD.
